(19) United States
(12) Patent Application Publication
(10) Pub. No.: US 2023/0170041 A1
(43) Pub. Date: Jun. 1, 2023

(54) Method and System for Predicting a Synergistic Effect

(71) Applicant: City University of Hong Kong, Hong Kong (CN)

(72) Inventors: Qingpeng ZHANG, Hong Kong (CN); Zhongzhi XU, Hong Kong (CN); Jiannan YANG, Hong Kong (CN)

(21) Appl. No.: 17/457,044

(22) Filed: Dec. 1, 2021

(51) Int. Cl. G16B 5/20; G16H 70/40

(52) U.S. Cl. CPC G16B 5/20; G16H 70/40

(57) ABSTRACT

A method, system, apparatus and a non-transitory computer-readable medium storing instruction thereon is provided for predicting a probability of a synergistic effect between a combination of drugs in the treatment of, for example, a complex disease, such as cancer, through the application of an end-to-end deep learning framework based on a protein-protein interaction (PPI) network, drug-protein associations and cell line-protein associations.

### FIELD OF THE INVENTION

The invention relates to a method, system, apparatus and a non-transitory computer-readable medium storing instruction thereon for predicting a synergistic effect. In particular, the invention relates to, but is not limited to, a method, system, apparatus and a non-transitory computer-readable medium storing instruction thereon configured predict the efficacy of a combination of drugs in the treatment of, for example, a complex disease such as, but not limited to, cancer.

### BACKGROUND TO THE INVENTION

Reference to background art herein is not to be construed as an admission that such art constitutes common general knowledge in Australia or elsewhere.

Drug combination therapy has shown great promise to improve the efficacy and extend the duration of response in the treatment of complex diseases, such as cancers, human immunodeficiency virus, and cardiovascular diseases. However, identifying synergistic drug combinations is challenging because the combinatorial space of drugs is huge, and the effects or side effects of any given treatment can be adverse. Therefore, effective identification of potential synergistic drug combinations for specific diseases, such as those identified above which can minimize the unexpected adverse effects and maximize the synergistic benefits, is a pressing need.

Traditional drug combination identification is typically based on clinical experience. With the development of High-Throughput Screening (HTS) technology, researchers may discover synergistic combinations by in vitro experiments. However, the cost in terms of money and time invested into such experiments can be prohibitive.

In contrast, in silico approaches, such as machine learning methods, offer an opportunity to explore a large combinatorial space efficiently. Existing in silico models, such as random forest and support vector machines, mainly focus on the drug's chemical features or biological targets of a specific cancer. Other recent deep learning models such as “DeepSynergy” (Preuer K, Lewis R P, Hochreiter S, Bender A, Bulusu K C, Klambauer G. DeepSynergy: predicting anti-cancer drug synergy with Deep Learning. Bioinformatics 2018; 34(9):1538-46) introduce further cancer genomic information to make predictions for multiple types of cancers. The most recent model, “AuDNNsynergy” (Zhang T, Zhang L, Payne P R, Li F. Synergistic Drug Combination Prediction by Integrating Multiomics Data in Deep Learning Models. Translational Bioinformatics for Therapeutic Development: Springer, 2021:223-38), integrates multi-omics data such as gene expression, copy number, and genetic mutation data of tumor samples by introducing three auto-encoders. These methods are all based on the assumption that drugs with similar chemical structures have similar treatment effects.

Other previously known methods such as Network Proximity (for example in Cheng F, Kovács I A, Barabási A-L. Network-based prediction of drug combinations. Nature communications 2019; 10(1):1-11), Matrix-Factorization (for example in Grarep: Learning graph representations with global structural information. Proceedings of the 24th ACM international on conference on information and knowledge management; 2015), Random Walk (for example Deepwalk: Online learning of social representations. Proceedings of the 20th ACM SIGKDD international conference on Knowledge discovery and data mining; 2014; and node2vec: Scalable feature learning for networks. Proceedings of the 22nd ACM SIGKDD international conference on Knowledge discovery and data mining; 2016) and Graph convolutional Networks—GCN (for example in Kipf T N, Welling M. Semi-supervised classification with graph convolutional networks. arXiv preprint arXiv:1609.02907 2016; and Kgnn: Knowledge graph neural network for drug-drug interaction prediction. Proceedings of the Twenty-Ninth International Joint Conference on Artificial Intelligence, IJCAI-20 (International Joint Conferences on Artificial Intelligence Organization); 2020) also have similar methods of operation.

These existing examples have been attempts to provide an accurate prediction, however they do not take into consideration the complex biological interactions among the proteins related to drugs and diseases, and thus lack the ability to explicitly capture other relevant factors such as the toxic effects resulting from combining drugs.

There is therefore a need for one or more of an improved in silico method, system, apparatus and/or non-transitory computer-readable medium storing instruction thereon for predicting synergistic effects.

### OBJECT OF THE INVENTION

It is a preferred object of this invention to provide one or more of a system, method, apparatus and/or non-transitory computer-readable medium storing instruction thereon for predicting a synergistic effect which overcomes or at least ameliorates one or more of the aforementioned disadvantages or problems with existing systems.

It is a further preferred object of the invention to provide one or more of a system, method, apparatus and/or non-transitory computer-readable medium storing instruction thereon including deep learning, namely a Graph Convolutional Network for Drug Synergy, to automatically identify synergistic drug combinations for, for example, specific cancer cell lines, with only the target proteins and related proteins of drugs and, for example, cancer cell lines as input.

Other preferred objects of the present invention will become apparent from the following description.

### SUMMARY OF INVENTION

In one form, although not the only form, the invention resides in a computer implemented method for predicting a synergistic effect between at least a first drug i and a second drug j in the treatment of a disease associated with a cell line k, the method comprising the steps:

obtaining a protein-protein interaction (PPI) network, drug-protein associations and cell line-protein associations;
extracting target proteins (g) in the PPI network for each e, where e includes i,j,k, from the drug-protein associations and/or the cell line-protein associations;
extending along edges in the PPI network from each target protein to form a radiant field (Sₑ^{h}) which contains proteins within h hops from each target protein;
determining an interaction field for each pair of i,j,k that relates to the union of their respective radiant fields; and
determining a probability of the synergistic effect based on the interaction fields.

Preferably, a graph convolutional network is used to determine the contribution of each target protein to the synergistic effect.

Preferably, the interaction fields are fed into an aggregation layer iteratively to obtain a latent representation of each of i,j,k.

Preferably, determining the probability of the synergistic effect is based on determining a therapy score and a toxicity score relating to the interaction fields.

Preferably, determining the therapy score and the toxicity score includes calculating the inner product of the representations of i,j,k to measure the similarity between each of i,j,k.

In another form, the invention resides in a system for predicting a synergistic effect between at least a first drug i and a second drug j in the treatment of a disease associated with a cell line k, the system comprising:

at least one input device for accessing a protein-protein interaction (PPI) network, drug-protein associations and cell line-protein associations;
at least one processor to perform the steps of:
extracting target proteins (Sₑ⁰) in the PPI network for each entity e, where e includes i,j,k, from the drug-protein associations and/or the cell line-protein associations;
extending along edges in the PPI network from each target protein to form a radiant field (Sₑ^{h}) which contains proteins within h hops from each target protein;
determining an interaction field for each pair of i,j,k that relates to the union of their respective radiant fields; and
determining a probability of the synergistic effect based on the interaction fields.

Preferably, a graph convolutional network is used in the system to determine the contribution of each target protein to the synergistic effect.

Preferably, the system includes an aggregation layer and the interaction fields are fed into an aggregation layer iteratively to obtain a latent representation of each of i,j,k.

Preferably, determining the probability of the synergistic effect by the system is based on determining a therapy score and a toxicity score relating to the interaction fields.

Preferably, determining the therapy score and the toxicity score by the system, includes calculating the inner product of the representations of i,j,k to measure the similarity between each of i,j,k.

In another form, the invention resides in, a non-transitory computer-readable medium storing instructions thereon, which when executed by a processor cause the processor to predict a synergistic effect between at least a first drug i and a second drug j in the treatment of a disease associated with a cell line k, the processor performing the steps:

obtaining a protein-protein interaction (PPI) network, drug-protein associations and cell line-protein associations;
extracting target proteins (g) in the PPI network for each e, where e includes i,j,k, from the drug-protein associations and/or the cell line-protein associations;
extending along edges in the PPI network from each target protein to form a radiant field (Sₑ^{h}) which contains proteins within h hops from each target protein;
determining an interaction field for each pair of i,j,k that relates to the union of their respective radiant fields; and
determining a probability of the synergistic effect based on the interaction fields.

Further features and advantages of the present invention will become apparent from the following detailed description.

### BRIEF DESCRIPTION OF THE DRAWINGS

By way of example only, preferred embodiments of the invention will be described more fully hereinafter with reference to the accompanying figures, wherein:

FIG. 1 illustrates a representation of a PPI network used in the invention;

FIG. 2 illustrates representations of each radiant field;

FIG. 3 illustrates a representation of the therapy score;

FIG. 4 illustrates a representation of toxic score;

FIG. 5 illustrates a schematic representation of a system according to an embodiment of the invention.

FIG. 6 summarises a set of results comparing the present invention to prior art methods.

FIG. 7 shows further results of the comparison shown in FIG. 6, the distribution of the prediction performance for drugs and cell lines.

FIG. 8 shows further results of the comparison shown in FIG. 6, the distribution of the number of related proteins of drugs and cell lines with their corresponding AUC-ROC values.

FIG. 9 shows further results of the comparison shown in FIG. 6, the distribution of the average degree of related proteins of drugs and cell lines with their corresponding AUC-ROC values.

FIG. 10 shows further results of the comparison shown in FIG. 6, the tissue-specific distribution of the AUC-ROC values for all cell lines.

FIG. 11 shows the top 20 most popular pivotal proteins among all the drugs and cell lines.

FIG. 12 shows the relationships between the occurrence frequencies among all entities with the degree of each pivotal protein.

FIG. 13 shows the molecular function and biological process for the top 20 most frequent pivotal proteins of drugs.

FIG. 14 shows the molecular function and biological process for the top 20 most frequent pivotal proteins of cell-lines.

FIG. 15 shows a visualization of contribution weights with respect to the related proteins for synergistic drug combination verified by clinical trials.

FIG. 16 shows the performance of variations of various embodiments of the invention on the DrugCombDB dataset.

FIG. 17 results of embodiments of the invention with respect to the depth in the interaction fields H and the depth in the interaction fields S′.

### DETAILED DESCRIPTION

In this specification, adjectives such as first and second, forward and backward, upward and downward, upper and lower, top and bottom and the like may be used solely to distinguish one element or action from another element or action without necessarily requiring or implying any actual such relationship or order. Where the context permits, reference to an integer or a component or step (or the like) is not to be interpreted as being limited to only one of that integer, component, or step, but rather could be one or more of that integer, component, or step etc.

In this specification, the terms ‘comprises’, ‘comprising’, ‘includes’, ‘including’, or similar terms are intended to mean a non-exclusive inclusion, such that a method, system or apparatus that comprises a list of elements does not include those elements solely, but may well include other elements not listed.

Most anti-cancer drugs work with specific proteins related to cancer cells in a Protein-Protein Interaction (PPI) network. Recent network science studies provide evidence that the topological relations between drugs and diseases in the PPI network play an essential role in drug identification. More specifically:
(a) An effective drug should target the proteins within or near the corresponding disease module; and
(b) Two drugs with synergistic effects should target complementary (non-overlapping) proteins to prevent the toxicities brought by over-exposure.

The potential predictability of considering the drug-drug and drug-disease relationships in the PPI network has been demonstrated. However, these network science methods only focus on the topological distance between proteins directly associated with drugs and diseases, while ignoring the local connections formed by neighbouring proteins and the global structure of the PPI network. In addition, existing network science approaches treat each protein homogeneously, whereas recent studies reveal that several proteins have a dominant contribution to the progression of cancers.

To address these challenges, the present invention provides an end-to-end machine learning framework, namely a Graph Convolutional Network for Drug Synergy, to identify synergistic drug combinations for specific cancer cell lines from the perspective of the molecular mechanism (i.e., biological interactions between proteins) in a PPI network. The invention introduces a Graph Convolutional Network (GCN) component to learn the rich topological information of drug and disease modules in the PPI network by extending neighbour aggregation to deeper layers. At each layer, the invention utilizes an attention component to determine the contribution of each protein and uses them to guide the aggregation. Then, the invention defines two scores to explicitly evaluate two pharmacological characteristics of drugs: a joint therapy score measured by the similarity between the drug combinations and the cancer cell lines; and a toxicity score measured by the similarity between two drugs.

Any suitable PPI network can be used, for instance the comprehensive human interactome network generated by Cheng et. al. (Cheng F, Kovács I A, Barabási A-L. Network-based prediction of drug combinations. Nature communications 2019; 10(1):1-11), which is assembled through 15 commonly used databases and experimental evidence. This particular PPI network contains 217,160 interactions connecting 15,790 unique proteins, with each protein mapped to its coding genes.

To determine reference points within the PPI for any given drug, a drug-protein association is required. Any suitable set of data can be used that maps particular drugs, to proteins they are effective at targeting.

Similarly, cell line-protein associations' are required to determine reference points within the PPI for a given cell line. Any suitable set of associations can be used, for instance the Cancer Cell Line Encyclopedia (Barretina J, Caponigro G, Stransky N, et al. The Cancer Cell Line Encyclopedia enables predictive modelling of anticancer drug sensitivity. Nature 2012; 483(7391):603-07), which has 18,022 genes mapped to their coding proteins in the PPI network, 1,035 cancer cell lines, and 74,9551 associations.

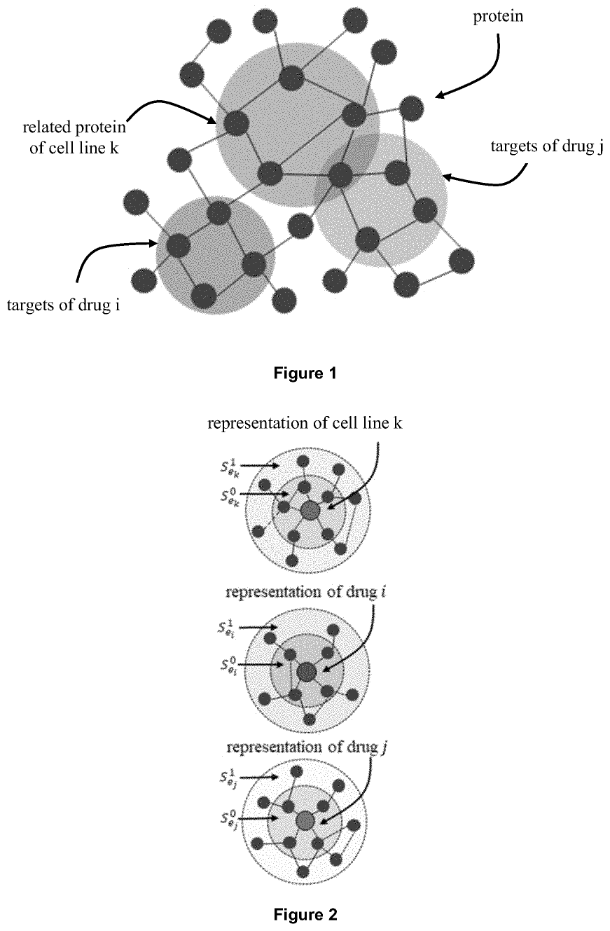

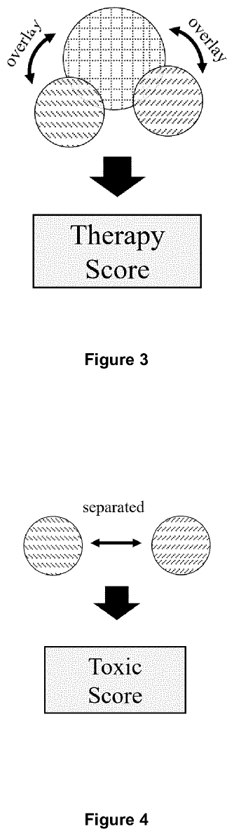

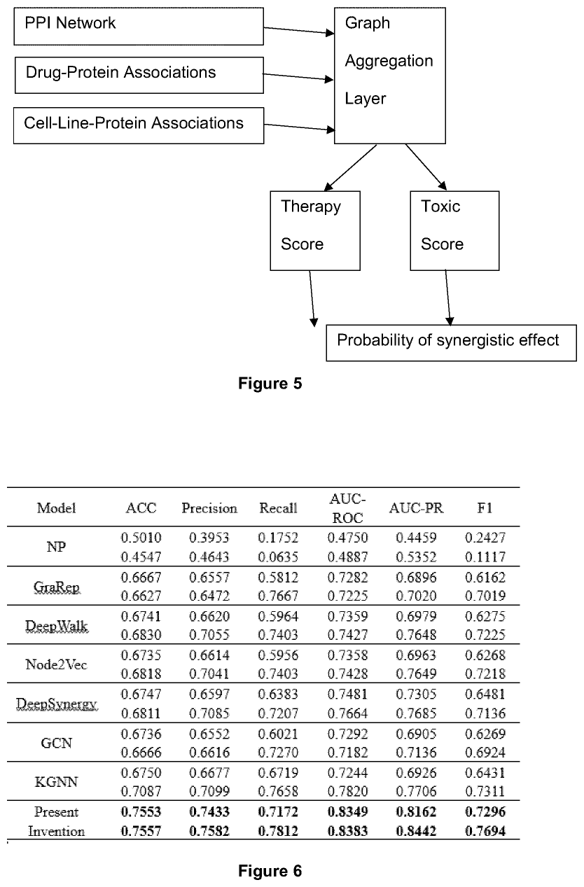

With reference to FIGS. 1 to 5, the invention takes a combination of drug i and drug j with a cell line k as input and outputs the predicted probability that the drug combination is synergistic to the corresponding cell line. For each entity e in the input (i, j, k), its directed connected proteins Sₑ⁰ are extracted from the drug-protein or cell line-protein associations as target field, and then extended along edges in the PPI network to form a radiant field Sₑ⁰(k=1, 2, . . . , H), which contains the proteins that are within k hops away from entity e. The radiant field captures the local interactions between the proteins that might play a role but are not directly targeted by the entity (either a drug or a cancer cell line).

The union of target and radiant field Sₑ^{k}(k=0, 1, 2, . . . , H) are defined as the interaction field of entity e and these fields are fed into the aggregation layer iteratively to obtain the latent representation of entity e. An attention mechanism is introduced to characterize the heterogeneous effect of proteins in the interaction field. The representations of drug i, drug j, and cell line k denote positions of their target modules in the PPI network, and they are combined to estimate the synergistic effect of the drug combination for the cancer cell line. Based on the relationships between the pharmacological effects with the topological positions of the related protein modules of drugs and cell lines, the synergistic effect is explicitly measured by two scores: therapy score and toxicity score.

Using a spatial-based GCN approach, the invention defines the set of H-hop relevant neighbors Sₑ^{h}(h=0, 1, 2, . . . H) of entity e as its interaction fields, which contains the target filed Sₑ⁰ (direct targets of entity e and the radiant field Sₑ^{h} (h=1, 2, . . . H) (indirectly connected proteins that may also play a role in the therapy mechanism). Note that the size of Sₑ^{h} may vary significantly across entities, thus we uniformly sample a fixed-size subset Ŝ instead of using all the proteins at each layer.

After defining the radiant fields and interaction fields the invention aggregates the information of proteins at each layer in the interaction field for one entity. This is achieved through Contribution propagation and an aggregation layer.

Using an attention mechanism, the invention takes the inner product (defined as g: R^{d}×R^{d}→R for simplicity) to compute the protein's contribution to the effect of the drug or the cell line. Given the representation of entity e and its interaction field Sₑ^{h}, each protein p∈Sₑ^{h} is assigned a contribution weight:

πₚ^{e}=g(e,p),

where e∈R^{d} and p∈R^{d}, d is the dimension of the representations. m denotes the contribution of a protein p to the effect of an entity e and can be regarded as the similarity of e and p.

After obtaining the contribution of each protein in one layer, the invention computes the linear combination of the proteins in this layer weighted by their corresponding contributions:

$${I}_{{S}_{e}^{h}} = {\sum }_{p ∈ {S}_{e}^{h}} {\hat{π}}_{p}^{e} p ,$$

where I_{S}_{ₑ}_{^{h}} is the representation of layer h and \hat{π}ₚ^{e} is the normalized contribution score:

$${\hat{π}}_{p}^{e} = \frac{exp ( {π}_{p}^{e} )}{{\sum }_{p ∈ {S}_{e}^{h}} exp ( {π}_{p}^{e} )} .$$

By updating the representation of entity e in the equations above using the representation I_{S}_{ₑ}_{^{h}} of interaction field Sₑ^{h}, we can repeat the procedure of contribution propagation to deeper layers in order to obtain entity e's multiple-hops' representations {I_{S}_{ₑ}_{⁰}, I_{S}_{ₑ}_{¹}, . . . , I_{S}_{ₑ}_{^{H}}}. Aggregating the representations of different layers can be achieved in several methods:

aggreₛᵤₘ=W·Σₕ₌₀^{H}I_{S}_{ₑ}_{^{h}}+b;

aggre_{concat}=W·concat(I_{S}_{ₑ}_{⁰},I_{S}_{ₑ}_{¹}, . . . ,I_{S}_{ₑ}_{^{H}})+b; or,

aggre_{neighbor}=W·I_{S}_{ₑ}_{^{h}}+b.

Previous experimentation has shown that aggre_{concat} performs optimally from these methods however it is envisaged that any of these methods could be utilised with the invention. The final representation of entity e is preferably:

ê=W_{agg}·concat(I_{S}_{ₑ}_{⁰},I_{S}_{ₑ}_{¹}, . . . ,I_{S}_{ₑ}_{^{H}})+b_{agg}, (4)

where ê is the final representation of entity e, W_{agg} and b_{agg} are the aggregation weight and bias, respectively.

Given the final representations êᵢ, ê₁ and êₖ of two drugs and one cell line, the invention predicts whether the drug combination is synergistic in this particular cell line. The invention uses the inner product of entities' representations to measure the similarity between two entities. The higher the similarity, the more overlap between the two corresponding protein modules. To this end, the invention defines two scores: Therapy score, and Toxic score.

The drugs targeting proteins that are within or near the proteins in the disease module are found to be more effective in treating the disease. Consequently, three methods (denoted as Γ: R^{d}×R^{d}× . . . R^{d}→R) are used to compute the therapy score sₚ, which is evaluated by the similarity between the final representations of drug pairs and cell lines.
Weighted inner product first computes the inner products of two drugs with the cell line separately and then takes the weighted sum as the therapy score.

Γ_{wip}(êᵢ,êⱼ,êₖ)=α(êᵢ⊙êⱼ)+β(êⱼ⊙êₖ),

where α and β are the weights, respectively.
Max pooling utilizes the element-wise maximum of the representations of two drugs as the combined drug representation, and then computes the inner product of the combined drug representation and the cell line representation.

Γₘₚ(êᵢ,êⱼ,êₖ)=max(êᵢ,êⱼ)⊙êₖ.
Transformation matrix concatenates the representations of the two drugs, and then computes the inner product of the concatenated drug representation and the cell line representation.

Γₜₘ(êᵢ,êⱼ,êₖ)=(W_{Γ}·concat(êᵢ,êⱼ)+b_{Γ})⊙êₖ,
where W_{Γ} and b_{Γ} are the weight and bias, respectively.

It is recognized that a combination of drugs that are overlapping in the PPI network should be avoided to prevent toxicities. Thus, the invention prefers a pair of drugs that are dissimilar. The toxicity score is computed as the inner product of the two drugs' representations.

sₙ=Ψ(êᵢ,êⱼ)=êᵢ⊙êⱼ.

Synergistic drug combination prediction can subsequently be viewed as a binary classification task. Given the representations of drug i, drug j, and cell line k, the synergistic probability ŷ_{i,j,k} is evaluated by the difference between the therapy score sₚ and the toxicity score sₙ.

ŷ_{i,j,k}=σ(sₚ−Sₙ)

where σ is the sigmoid function.

Given a set of drug-drug-cell trios, the invention formulates the following loss function for the invention:

$$𝕃 = {\sum }_{( i , j , k ) ∈ Y ( i \neq j )} ℒ ( {y}_{i , j , k} {\hat{y}}_{i , j , k} ) + \frac{{λ}_{1}}{2} {‖ 𝔼 ‖}_{2}^{2} + \frac{{λ}_{2}}{2} {‖ Θ ‖}_{2}^{2} ,$$

where [symbol] (y_{i,j,k}, ŷ_{i,j,k})=−y_{i,j,k} log ŷ_{i,j,k}−(1−y_{i,j,k}) log(1−ŷ_{i,j,k}) is the binary-cross-entropy loss. The second and third terms are the regularizes to prevent over-fitting, where [symbol] is the embedding matrix for all items.

As an example of the efficacy of an embodiment of the invention, a comparison was made between an embodiment of the invention and a number of prior art methods based on Network Proximity, Matrix Factorization, Random Walk, Deep Neural Network (DNN), and Graph Convolutional Network (GCN). This comparison measured five metrics to evaluate efficacy: accuracy (ACC), precision, recall, area under the receiver operating characteristic curve (AUC-ROC), area under precision-recall curve (AUC-PR), and F1 score.

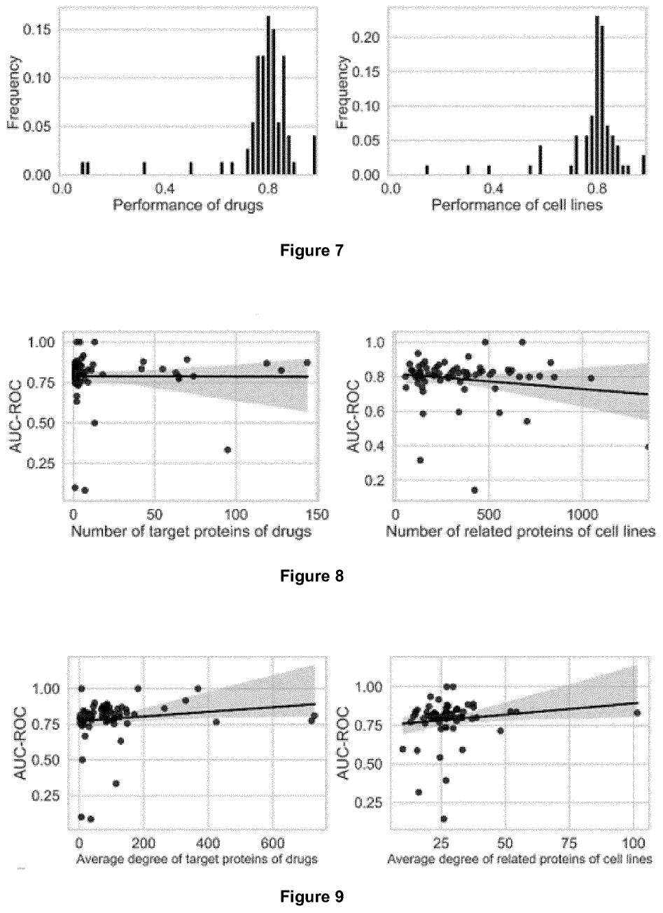

FIG. 6 shows the results of the abovementioned comparison. The first/second row of each tested method corresponds to results reported on the DrugCombDB dataset and Oncology-Screen dataset respectively. Each result is derived from each method being repeated 5 times and the average output reported. In summary, an embodiment of the invention significantly outperformed the prior art methods. This demonstrates that present invention can predict the synergistic drug combinations for cancers well by capturing the topological relations between drug combination and cell line within the PPI network.

More specifically, as a model specific-designed for the Drug-Drug Interaction prediction task, KGNN has a graph embedding module with similarities to the present invention, but it underperforms the present invention significantly (e.g., at least 15.25% AUC-ROC reduction on DrugCombDB), indicating that incorporating the topological proximity to characterize the therapy and toxicity effects can greatly improve prediction power.

Network Proximity (NP), which is based on proximity measures (e.g., z-score or separation score), shows mild prediction ability, but its performance is the least compared with other machine-learning-based models, indicating that simple proximity measures cannot fully capture the complex high-dimensional relations between drugs and cancer cell lines.

The performances of random-walk-based models are nearly identical on both datasets. They also outperform the matrix-factorization-based model (GraRep). This is because deep walks can capture sufficient structural information on these relatively smaller datasets. If the networks are large (such as social and bibliographic networks), GCN-based methods could have performed better than random-walk-based methods.

KGNN performs better than GCN, indicating that incorporating the attention mechanism is helpful in learning the relations between drugs and cancer cell lines. The present invention is the only model designed specifically for drug combination identification. The performance of the present invention, though better than that of the basic GCN, is lower than that of KGNN, indicating that the incapability of capturing the topological information might have hindered its prediction power.

FIG. 7 demonstrates performance results categorised by entity, either cell-line or drug, on the DrugCombDB dataset, and shows that the performance varies across this domain. The performance is represented by the AUC-ROC value between all the observed and the predicted combinations in the test set. As will be recognised, the present invention achieves great prediction ability (AUC-ROC is larger than 0.75) for more than 85% of drugs and 90% of cell lines.

The number and degree of related proteins vary dramatically among drugs and cell lines. The Pearson correlation between the number of related proteins and the prediction performance (represented in FIG. 8), as well as the Pearson correlation between the average degree and the AUC-ROC of predictions (represented in FIG. 9) show that the correlations are insignificant for both drugs and cell lines, indicating that simple statistics of the network connectivity are not predictive. In contrast, the complex biological mechanism and unique pharmacological properties are well captured by the present invention model.

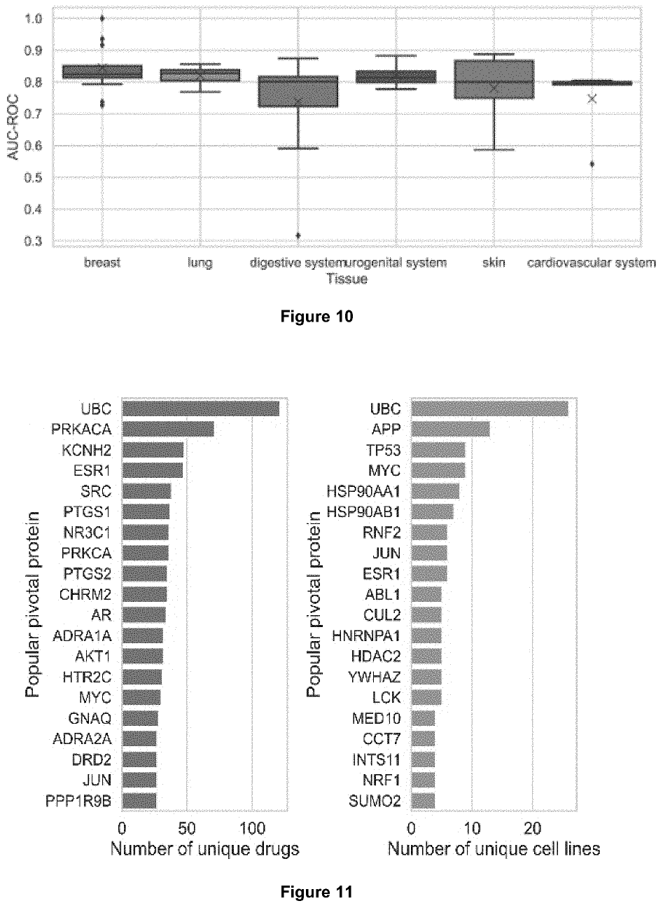

Prediction performance can also be characterised by tissue type. Across six types of tissues, shown in FIG. 10, in general, the performance of the present invention does not vary dramatically across different tissues, showing the generalization power of the present invention, which can be introduced to different tissues. More specifically, the present invention achieves high AUC-ROC values with small variance in lung (median, 0.8284), breast (median, 0.8243), and urogenital system (median, 0.8135). In contrast, for digestive system (median, 0.8010) and skin (median, 0.8000), there is a relatively larger variance compared with other 3 tissues, and relatively smaller AUC-ROC values. For cardiovascular system, the performance on this tissue does not vary too much (relatively smaller variance) but achieves a relatively smaller median AUC-ROC value (0.7983).

The present invention may apply an attention mechanism to automatically allocate contribution weights to the proteins that are related/targeted by drugs and cell lines. This weight represents the contribution to the synergistic prediction of the present invention. By defining pivotal proteins as the top 10 proteins by the contribution weight for each drug and cell line, the roles played by these pivotal proteins in the connectivity of the PPI network, as well as their biological mechanisms, can be examined.

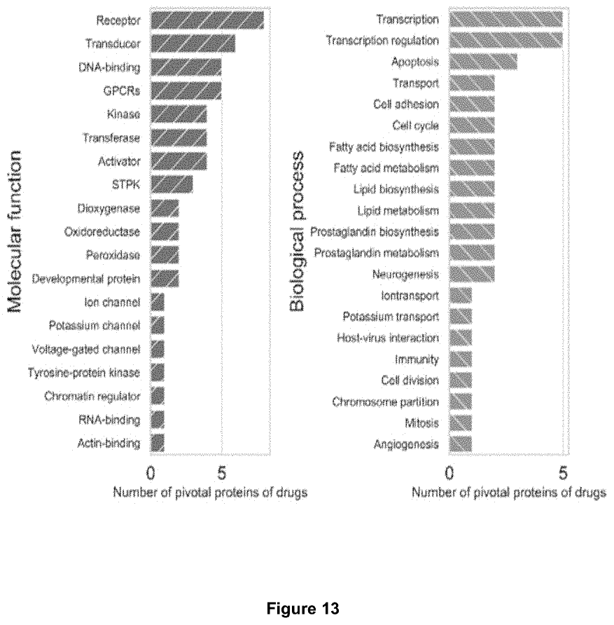

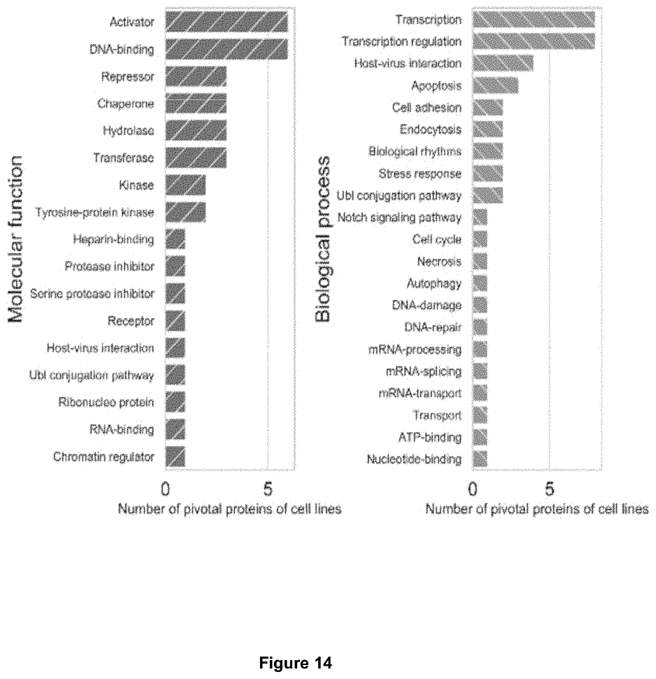

FIG. 11 presents the top 20 proteins measured by the frequency of being the pivotal protein for drugs (left of FIG. 11) and cell lines (right of FIG. 11). They are significantly different, with only four common proteins: UBC, ESR1, JUN, and MYC. They represent the most important proteins in the progress of some cancers. For instance, UBC, which encodes ubiquitin C contributes to the regulation of many cellular events, such as innate immunity, DNA repair and kinase activity through the ubiquitin-proteasome pathway. There is a synthetic lethal relationship between UBB and UBC that has potential to be exploited as a therapeutic strategy to fight these devastating cancers. The molecular functions and biological processes of these popular pivotal proteins have differences between those for drugs and those for cell lines (as shown in FIGS. 13 and 14). Specifically, the popular pivotal proteins for drugs are more often acted as receptor and transducer, while those for cell lines tend to be activator and DNA-binding in view of molecular functions. Considering roles in the biological process, the popular proteins for both drugs and cell lines tend to play a role in transcription and transcription regulation.

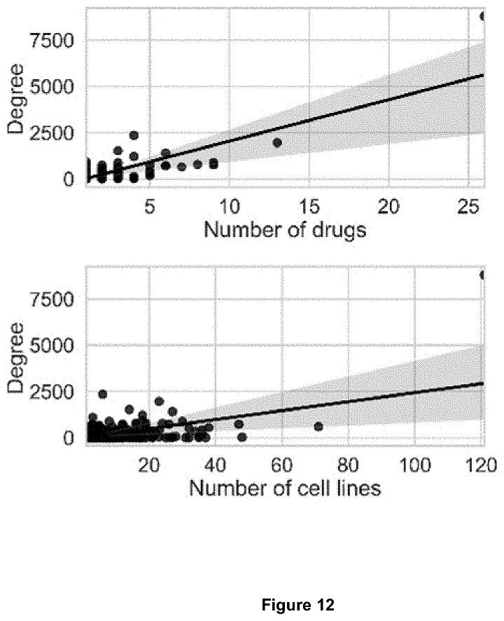

There is a question as to whether network connectivity is associated with the pivotal role. By calculating the Pearson correlation between the degree and the frequency of pivotal proteins in drugs (FIG. 12 upper) and in cell lines (FIG. 12 bottom), respectively this question can be answered. The results show that positive correlations in both drugs (0.7947, p-value <0.0001) and cell lines (0.5585, p-value <0.0001) exist. These results indicate that the proteins that interact with many others tend to be pivotal proteins. This is aligned with the previous findings that a high degree of protein is associated with its importance in the biological mechanism in the human body.

To intuitively demonstrate the efficacy of the present invention, clinically-verified drug combinations can be included in the testing set. For instance: the synergistic combination of Pemetrexed with Crizotinib on cell line NCIH322; and the synergistic combination of Pemetrexed with Gefitinib on cell line NCIH522. All these drugs are previously identified to be effective for the treatment to non-small cell lung cancer (NSCLC). For both combinations, the present invention generates positive predictions, and even though the combination of Pemetrexed with Gefitinib on NCIH522 in the dataset (DrugCombDB) appears as antagonistic, the present invention can still identify their previously known and clinically verified pharmacological effect.

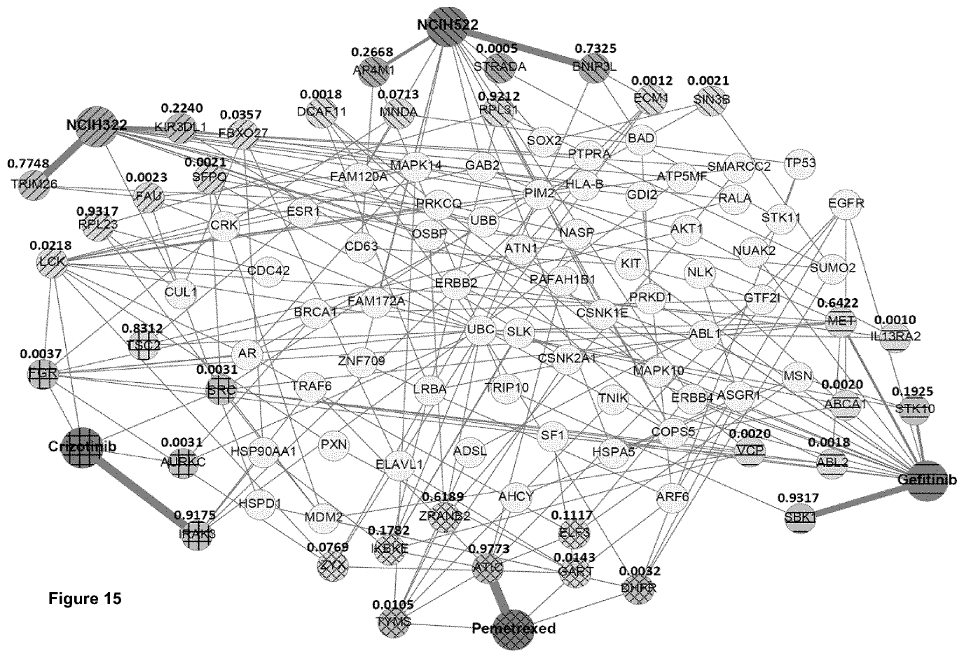

In the representation in FIG. 15, for each protein in the interaction field of one drug or cell line, the normalized contribution weights are directly obtained. Only proteins with significant contribution weights (i.e., more than 0.001) and the proteins connecting to them are visualized. In FIG. 15, such proteins are indicated by circles comprising no lines or cross-hatching. Two cell lines NCIH322 and NCIH522 of non-small-cell lung cancer (NSCLC) are marked with circles comprising diagonal lines right-to-left (top to bottom) and diagonal lines left-to-right (top to bottom), respectively, and the proteins related to them are marked with circles comprising diagonal lines of the same slope with corresponding lighter shading. The level of shading is proportional to the proximity (as measured by the inverse of the topological distance). For example, TRIM26 and KIR3DL1 are proteins related to NCIH322 and AP4M1, STRADA and BNIP3L are examples of proteins related to NCIH522. DCAF11, MNDA and RPL31 are further examples of proteins related to NCIH22, but their representative circles have a lighter shading indicating proportionally less proximity to the cell line NCIH522. Similarly, three drugs (Crizotinib, Pemetrexed, and Gefitinib) and their related proteins are marked with circles comprising vertical and horizontal cross-hatching, diagonal cross-hatching and horizontal lines, respectively. FIG. 15 shows that even though the targets of the three drugs mentioned do not directly connect to the targets of NCIH322 or NCIH522, the present invention still generates positive predictions based on their influential fields (indirectly connected proteins). Among hundreds of related proteins of NSCLC (NCIH322 and NCIH522), BNIP3L, TRIM26, AP4M1, RPL23 and so on are given a high priority by the present invention. Recent clinical and experimental evidence showed the close relationships of such proteins with NSCLC. For example, TRIM26 was decreased in NSCLC and overexpression of TRIM26 inhibited NSCLC cell growth by suppressing PI3K/AKT pathway, which suggested that TRIM26 could be a potential target for the treatment of NSCLC; the loss of BNIP3L regulation through p53 under hypoxia facilitates microenvironmental adaptation and maybe a key step in tumor development.

On the other hand, these three drugs, MET, SBK1, IRAK3, ATIC, IKBKE and so on are given high importance, which are all connected to the related proteins of two cell lines. Previous studies show that these proteins are highly related to the progression of NSCLS. For instance, MET is found to be a promising therapeutic target in advanced NSCLC, which can initiate and maintain tumor transformation, promote cell proliferation, survival, tumor invasion and angiogenesis when signals are abnormally activated; Target IKBKE has shown to be a strategy to eradicate EGFR-TKI-resistant NSCLC.

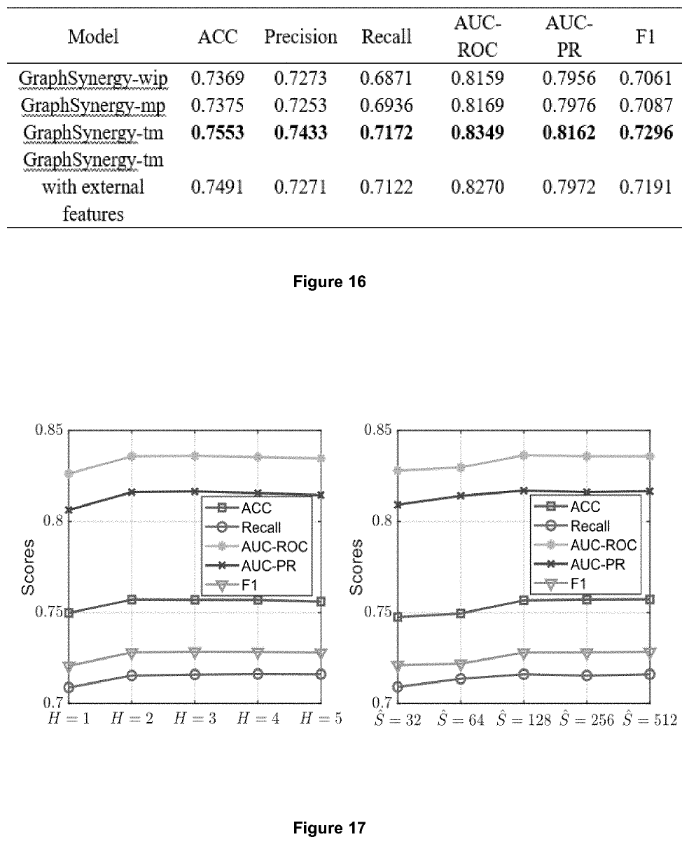

The above results demonstrate that the present invention can accurately identify synergistic anti-cancer combinations by capturing the biological mechanisms of proteins related to drugs and cancer cell lines in the PPI network. The performance of variants of the present invention are shown in FIGS. 16 and 17 (GraphSynergy-wip, GraphSynergy-mp and GraphSynergy-tm). Among three measures of therapy score, the present invention with transformation matrix (GraphSynergy-tm) performs the best, indicating that transformation matrix better captures the relations among the representations of drugs and cancer cell lines. As for the parameter sensitivity, we examine the effects of H, the depth in the interaction fields, and S\hat{ }, the sample size of neighbors in each layer. FIG. 17. shows the performance given other parameters are fixed. In terms of H, the present invention achieves the best performance when H≥2, indicating that a moderate depth is sufficient to capture the proteins that are most relevant to drug and cell line targets. In terms of S\hat{ }, we find that S\hat{ }=128 yields the best performance, indicating that the present invention needs to sample a representative subset of proteins in each layer to capture the complex relations between proteins in the PPI network.

The superior performance of the present invention suggests that the combination of network science knowledge with deep learning methods is a valuable tool for the discovery of efficacious anti-drug combinations. With enough biological knowledge of gene expressions or target proteins in the PPI network, the present invention is able to accurately identify novel combination therapies for multiple complex diseases.

The above description of various embodiments of the present invention is provided for purposes of description to one of ordinary skill in the related art. It is not intended to be exhaustive or to limit the invention to a single disclosed embodiment. As mentioned above, numerous alternatives and variations to the present invention will be apparent to those skilled in the art of the above teaching. Accordingly, while some alternative embodiments have been discussed specifically, other embodiments will be apparent or relatively easily developed by those of ordinary skill in the art. The invention is intended to embrace all alternatives, modifications, and variations of the present invention that have been discussed herein, and other embodiments that fall within the spirit and scope of the above-described invention.

1. A computer implemented method for predicting a synergistic effect between at least a first drug i and a second drug j in the treatment of a disease associated with a cell line k, the method comprising the steps:
a) obtaining a protein-protein interaction (PPI) network, drug-protein associations and cell line-protein associations;
b) extracting target proteins (Sₑ⁰) in the PPI network for each e, where e includes i,j,k, from the drug-protein associations and/or the cell line-protein associations;
c) extending along edges in the PPI network from each target protein to form a radiant field (Sₑ^{h}) which contains proteins within h hops from each target protein;
d) determining an interaction field for each pair of i,j,k that relates to the union of their respective radiant fields; and
e) determining a probability of the synergistic effect based on the interaction fields.

2. The computer implemented method of claim 1, wherein a graph convolutional network is used to determine the contribution of each target protein to the synergistic effect.

3. The computer implemented method of claim 1, wherein the interaction fields are fed into an aggregation layer iteratively to obtain a latent representation of each of i,j,k.

4. The computer implemented method of claim 1, wherein determining the probability of the synergistic effect is based on determining a therapy score and a toxicity score relating to the interaction fields.

5. The computer implemented method of claim 4, wherein determining the therapy score and the toxicity score includes calculating the inner product of the representations of i,j,k to measure the similarity between each of i,j,k.

6. The computer implemented method of claim 4, wherein a transformation matrix is applied to the therapy score to determine the probability of the synergistic effect.

7. The computer implemented method of claim 4, wherein a weighted inner product is applied to determine the therapy score.

8. The computer implemented method of claim 1, wherein maximum pooling is implemented to determine the synergistic effect.

9. A system for predicting a synergistic effect between at least a first drug i and a second drug j in the treatment of a disease associated with a cell line k, the system comprising:
at least one input device for accessing a protein-protein interaction (PPI) network, drug-protein associations and cell line-protein associations;
at least one processor to perform the steps of:
a) extracting target proteins (Sₑ⁰) in the PPI network for each entity e, where e includes i,j,k, from the drug-protein associations and/or the cell line-protein associations;
b) extending along edges in the PPI network from each target protein to form a radiant field (Sₑ^{h}) which contains proteins within h hops from each target protein;
c) determining an interaction field for each pair of i,j,k that relates to the union of their respective radiant fields; and
d) determining a probability of the synergistic effect based on the interaction fields.

10. The system of claim 9, wherein a graph convolutional network is used to determine the contribution of each target protein to the synergistic effect.

11. The system of claim 9, wherein the interaction fields are fed into an aggregation layer iteratively to obtain a latent representation of each of i,j,k.

12. The system of claim 9, wherein determining the probability of the synergistic effect is based on determining a therapy score and a toxicity score relating to the interaction fields.

13. The system of claim 12, wherein determining the therapy score and the toxicity score includes calculating the inner product of the representations of i,j,k to measure the similarity between each of i,j,k.

14. A non-transitory computer-readable medium storing instructions thereon, which when executed by a processor cause the processor to predict a synergistic effect between at least a first drug i and a second drug j in the treatment of a disease associated with a cell line k, the processor performing the steps:
a) obtaining a protein-protein interaction (PPI) network, drug-protein associations and cell line-protein associations;
b) extracting target proteins (Sₑ⁰) in the PPI network for each e, where e includes i,j,k, from the drug-protein associations and/or the cell line-protein associations;
c) extending along edges in the PPI network from each target protein to form a radiant field (Sₑ^{h}) which contains proteins within h hops from each target protein;
d) determining an interaction field for each pair of i,j,k that relates to the union of their respective radiant fields; and
e) determining a probability of the synergistic effect based on the interaction fields.

15. The non-transitory computer-readable medium of claim 14, wherein a graph convolutional network is used to determine the contribution of each target protein to the synergistic effect.

16. The non-transitory computer-readable medium of claim 14, wherein the interaction fields are fed into an aggregation layer iteratively to obtain a latent representation of each of i,j,k.

17. The non-transitory computer-readable medium of claim 14, wherein determining the probability of the synergistic effect is based on determining a therapy score and a toxicity score relating to the interaction fields.

18. The non-transitory computer-readable medium of claim 17, wherein determining the therapy score and the toxicity score includes calculating the inner product of the representations of i,j,k to measure the similarity between each of i,j,k.
